(19) United States
(12) Patent Application Publication
(10) Pub. No.: US 2023/0012936 A1
(43) Pub. Date: Jan. 19, 2023

(54) COMBINATION THERAPY USING GLUCAGON AND GLP-1 CO-AGONISTS FOR THE TREATMENT OF OBESITY

(71) Applicant: MEDIMMUNE, LLC, GAITHERSBURG, MD (US)

(72) Inventors: ARMANDO FLOR, GAITHERSBURG, MD (US); PHILIP AMBERY, CAMBRIDGE (GB)

(21) Appl. No.: 17/781,494

(22) PCT Filed: Dec. 1, 2020
(86) PCT No.: PCT/US2020/062695

§ 371 Date: Jun. 1, 2022

Related U.S. Application Data
(60) Provisional application No. 62/943,014, filed on Dec. 3, 2019.

(51) Int. Cl. A61K 38/26; A61K 31/155; A61K 31/7034; A61P 3/04; A61P 3/10; C07K 14/605

(52) U.S. Cl. CPC A61K 38/26; A61K 31/155; A61K 31/7034; A61P 3/04; A61P 3/10; C07K 14/605

(57) ABSTRACT

Provided herein are methods of improving glycemic control, reducing weight, and/or treating type 2 diabetes mellitus in human patients comprising administering GLP-1/glucagon agonist peptides, dapagliflozin, and metformin.

### REFERENCE TO SEQUENCE LISTING SUBMITTED ELECTRONICALLY

The content of the electronically submitted sequence listing in ASCII text file (Name: Sequencelisting_ST25.txt; Size: 1,763 bytes; and Date of Creation: Dec. 2, 2019) filed with the application is incorporated herein by reference in its entirety.

### BACKGROUND

The incidence of obesity and diabetes have been rising in epidemic proportions. Diabetes is characterized by high levels of blood glucose resulting from defects in insulin production, insulin action, or both. Type 2 diabetes mellitus (T2DM) accounts for some 90 to 95 percent of all diagnosed cases of diabetes, and the risk of type 2 diabetes rises with increasing body weight. The prevalence of type 2 diabetes is three to seven times higher in those who are affected by obesity than in normal weight adults, and is 20 times more likely in those with a body mass index (BMI) greater than 35 kg/m². However, weight-loss can improve, control or cure type 2 diabetes.

Both glucagon and GLP-1, acting as agonists at their respective receptors, have been shown to be effective in weight loss. Certain GLP-1 analogs are being sold or are in development for treatment of obesity including, e.g., Liraglutide (VICTOZA® from Novo Nordisk) and Exenatide (Byetta® from Eli Lilly/Amylin). Glucagon/GLP-1 agonist peptides have also been disclosed in WO 2014/091316.

While some therapies are available for the control of blood glucose, none currently achieve substantial weight loss, which remains a significant unmet need for patients. Fifty percent of patients progress from oral monotherapy for glucose control (usually with metformin) to initiation of insulin within 10 years, often taking multiple oral combination therapies before initiating insulin. The use of insulin exacerbates weight gain, which can be as great as 6 kg in the first year after starting insulin therapy. This weight gain can lead to increased insulin resistance, which is associated with hypertension, dyslipidemia, and an increased risk of major adverse cardiovascular events. With respect to reducing insulin resistance, significant weight loss (>5%) is the optimal intervention to reduce insulin resistance, although this can only be achieved reliably at present through intensive dietary and lifestyle interventions and/or bariatric surgery.

The combination of dapagliflozin and metformin as dual therapy has been extensively studied in T2DM subjects and has demonstrated a favorable benefit-risk profile. However, effects on HbA1c and weight over 52 weeks are modest (−0.52% in HbA1c from a baseline of 7.7%, and weight loss of about 3 kg). Over a longer period, larger effects may be optimal for disease modification.

Accordingly, there remains a need for effective methods of improving glycemic control, reducing weight, and treating type 2 diabetes mellitus (T2DM) in human patients that avoid adverse effects.

### BRIEF SUMMARY

Provided herein are methods of improving glycemic control, reducing weight, and treating type 2 diabetes mellitus (T2DM) in human patients comprising administering GLP-1/glucagon agonist peptides, dapagliflozin, and metformin.

Provided herein is a method of improving glycemic control in a human patient in need thereof, the method comprising administering to the patient a sufficient amount of (i) cotadutide (SEQ ID NO:4); (ii) dapagliflozin; and (iii) metformin to improve glycemic control.

Provided herein is a method of reducing body weight in a human patient in need thereof, the method comprising administering to the patient a sufficient amount of (i) cotadutide (SEQ ID NO:4); (ii) dapagliflozin; and (iii) metformin to reduce body weight.

Provided herein is a method of treating type 2 diabetes mellitus (T2DM) in a human patient in need thereof, the method comprising administering to the patient a sufficient amount of (i) cotadutide (SEQ ID NO:4); (ii) dapagliflozin; and (iii) metformin to treat T2DM.

In some aspects, the cotadutide is administered at an initial dose of at least 20 μg daily and administered at a second higher dose thereafter. In some aspects, the cotadutide is administered at a third dose after the administration of the second dose, wherein the third dose is higher than the second dose, optionally wherein the third dose does not exceed 600 μg daily or wherein the third dose does not exceed 300 μg daily. In some aspects, the initial dose is administered for about 7 days to about 14 days.

In some aspects, the cotadutide is administered at an initial dose of 100 μg daily for 7 days, at a second dose of 200 μg daily for the next 7 days, and subsequently at a dose of 300 μg daily. In some aspects, the cotadutide is administered by injection, optionally wherein the administration is subcutaneous.

In some aspects, the dapagliflozin is administered at a dose of 5 mg or 10 mg daily, optionally at a dose of 10 mg daily. In some aspects, the dapagliflozin is administered orally.

In some aspects, the metformin is administered at a dose of 500 mg to 2550 mg daily, 500 mg to 2000 mg daily, 500 mg to 1000 mg daily, or 500 mg to 850 mg daily. In some aspects, the metformin is administered orally.

In some aspects, the administration reduces the mixed-meal tolerance test (MMTT) plasma glucose area under the curve (AUC)₀₋₄ₕₒᵤᵣₛ in the patient. In some aspects, the administration reduces the MMTT plasma glucose AUC₀₋₄ₕₒᵤᵣₛ in the patient by at least 25 mg-hr/dL, at least 50 mg-hr/dL, at least 75 mg-hr/dL, at least 100 mg-hr/dL, or at least 150 mg-hr/dL. In some aspects, the administration reduces the percent MMTT plasma glucose AUC₀₋₂₄ hours in the patient by at least 5%, at least 10%, at least 15%, or at least 20%.

In some aspects, the administration reduces continuous glucose monitoring (CGM) glucose AUC₀₋₂₄ in the patient. In some aspects, the administration reduces CGM glucose AUC₀₋₂₄ in the patient by at least 200 mg-hr/dL, at least 250 mg-hr/dL, at least 300 mg-hr/dL, at least 350 mg-hr/dL, at least 400 mg-hr/dL, at least 450 mg-hr/dL, at least 500 mg-hr/dL, at least 550 mg-hr/dL, at least 600 mg-hr/dL, or at least 650 mg-hr/dL.

In some aspects, the administration reduces 24-hour CGM mean glucose in the patient. In some aspects, the administration reduces 24-hour CGM mean glucose in the patient by at least 10 mg/dL, at least 15 mg/dL, at least 20 mg/dL, or at least 25 mg/dL.

In some aspects, the administration reduces the standard deviation (SD) in CGM glucose in the patient by at 5 least mg/dL.

In some aspects, the administration reduces the CGM mean amplitude of glucose excursion (MAGE) in the patient by at least 10 mg/dL, at least 15 mg/dL, at least 20 mg/dL, or at least 25 mg/dL.

In some aspects, the administration reduces fasting plasma glucose (FPG) in the patient. In some aspects, the administration reduces FPG in the patient by at least 5 mg/dL, at least 10 mg/dL, at least 15 mg/dL, at least 20 mg/dL, at least 25 mg/dL, or at least 30 mg/dL.

In some aspects, the administration reduces Hemoglobin A1c (HbA1c) in the patient by at least 0.5% or by at least 1%.

In some aspects, the administration reduces body weight of the patient by at least 2 kg or by at least 3 kg.

In some aspects, the reduction occurs with 28 days from the initial administration of the cotadutide.

In some aspects, the administration produces euglycemic glucose levels in the patient.

In some aspects, the administration prevents hyperglycemic glucose levels in the patient.

In some aspects, the administration improves glycemic control in the patient.

In some aspects, the administration reduces body weight in the patient.

In some aspects, the administration treats T2DM in the patient.

In some aspects, the administration is for at least four weeks.

In some aspects, the administration is an adjunct to diet and exercise.

In some aspects, the patient has a body mass index (BMI) of ≥25 kg/m² to ≤40 kg/m². In some aspects, the patient has a hemoglobin A1c (HbA1c) of ≥7.0% to ≤10.0%. In some aspects, the patient has T2DM.

### BRIEF DESCRIPTION OF THE DRAWINGS/FIGURES

FIG. 1 shows the chemical structure, chemical formula (C₁₆₇H₂₅₂N₄₂O₅₅), and molecular weight (3728.09), for cotadutide (MEDI0382; SEQ ID NO:4).

FIG. 2 provides a flow diagram of the study of cotadutide (“MEDI0382”) in patients receiving dapagliflozin and metformin dual therapy. (See Example 1.)

### DETAILED DESCRIPTION

### I. Definitions

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. A peptide “comprising” a particular amino acid sequence refers to a peptide containing the amino acid sequence, wherein the peptide may or may not contain additional amino acids or other modifications to the amino acid sequence. A peptide “consisting of” a particular amino acid sequence refers to a peptide containing only the amino acid sequence and no additional amino acids or other modifications to the amino acid sequence. A peptide “comprising” an amino acid sequence “consisting of” a particular amino acid sequence refers to a peptide containing the amino acid sequence and no additional amino acids; however, the peptide may comprise other modifications to the amino acid sequence (e.g., an acyl moiety or a palmitoyl moiety).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids.

More specifically, the term “peptide” as used herein encompasses full length peptides and fragments, variants or derivatives thereof, e.g., a GLP-1/glucagon agonist peptide (e.g., 29, 30, or 31 amino acids in length). A “peptide” as disclosed herein, e.g., a GLP-1/glucagon agonist peptide, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

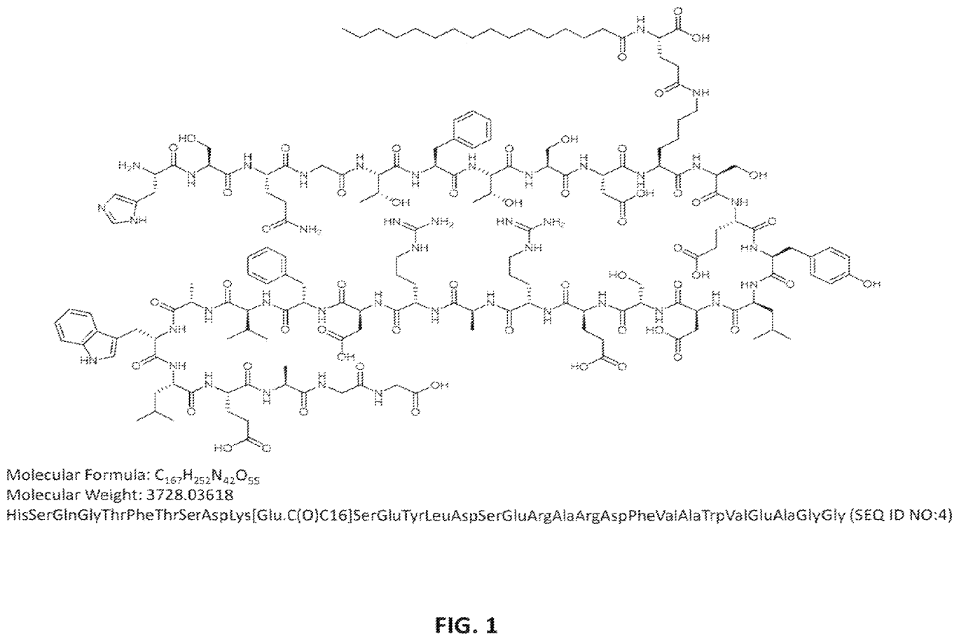

The terms “cotadutide” and “MEDI0382” are used herein to refer to a peptide with the structure shown in FIG. 1.

The term “isolated” refers to the state in which peptides or nucleic acids, will generally be in accordance with the present disclosure. Isolated peptides and isolated nucleic acids will be free or substantially free of material with which they are naturally associated such as other peptides or nucleic acids with which they are found in their natural environment, or the environment in which they are prepared (e.g., cell culture) when such preparation is by recombinant DNA technology practiced in vitro or in vivo. Peptides and nucleic acid can be formulated with diluents or adjuvants and still for practical purposes be isolated—for example the peptides will normally be mixed with gelatin or other carriers if used to coat microtitre plates for use in immunoassays, or will be mixed with pharmaceutically acceptable carriers or diluents when used in diagnosis or therapy.

A “recombinant” peptide refers to a peptide produced via recombinant DNA technology. Recombinantly produced peptides expressed in host cells are considered isolated for the purpose of the present disclosure, as are native or recombinant polypeptides which have been separated, fractionated, or partially or substantially purified by any suitable technique.

The terms “fragment,” “analog,” “derivative,” or “variant” when referring to a GLP-1/glucagon agonist peptide include any peptide which retains at least some desirable activity, e.g., binding to glucagon and/or GLP-1 receptors. Fragments of GLP-1/glucagon agonist peptides provided herein include proteolytic fragments, deletion fragments which exhibit desirable properties during expression, purification, and/or administration to a subject.

The term “variant,” as used herein, refers to a peptide that differs from the recited peptide due to amino acid substitutions, deletions, insertions, and/or modifications. Variants can be produced using art-known mutagenesis techniques. Variants can also, or alternatively, contain other modifications— for example a peptide can be conjugated or coupled, e.g., fused to a heterologous amino acid sequence or other moiety, e.g., for increasing half-life, solubility, or stability. Examples of moieties to be conjugated or coupled to a peptide provided herein include, but are not limited to, albumin, an immunoglobulin Fc region, polyethylene glycol (PEG), and the like. The peptide can also be conjugated or produced coupled to a linker or other sequence for ease of synthesis, purification or identification of the peptide (e.g., 6-His), or to enhance binding of the polypeptide to a solid support.

The terms “composition” or “pharmaceutical composition” refer to compositions containing a GLP-1/glucagon agonist peptide provided herein, along with e.g., pharmaceutically acceptable carriers, excipients, or diluents for administration to a subject in need of treatment, e.g., a human subject in need of improved glycemic control, weight loss, and/or treatment of T2DM.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

An “effective amount” is that amount of an agent provided herein (e.g., a GLP-1/glucagon agonist peptide, dapagliflozin, and/or metformin), the administration of which to a subject, either in a single dose or as part of a series, is effective for treatment, e.g., for improved glycemic control, weight loss, and/or treatment of T2DM.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

As used herein, a “subject in need thereof” or a “patient in need thereof” refers to an individual for whom it is desirable to treat, e.g., a subject in need of improved glycemic control, weight loss, and/or treatment of T2DM.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented.

Administration “in combination with” one or more further therapeutic agents includes simultaneous (concurrent) or consecutive administration in any order.

Terms such as “decreasing the severity” refer to therapeutic measures that slow down or lessen the symptoms of a diagnosed pathologic condition or disorder.

As used herein a “GLP-1/glucagon agonist peptide” is a chimeric peptide that exhibits activity at the glucagon receptor of at least about 1%, 5%, 10%, 20%, 30%, 40%, 50%, 60%, 70%, 80%, 90%, 95%, or more relative to native glucagon and also exhibits activity at the GLP-1 receptor of about at least about 1%, 5%, 10%, 20%, 30%, 40%, 50%, 60%, 70%, 80%, 90%, 95%, or more relative to native GLP-1.

As used herein the term “native glucagon” refers to naturally-occurring glucagon, e.g., human glucagon, comprising the sequence of SEQ ID NO: 1. The term “native GLP-1” refers to naturally-occurring GLP-1, e.g., human GLP-1, and is a generic term that encompasses, e.g., GLP-1(7-36) amide (SEQ ID NO:2), GLP-1(7-37) acid (SEQ ID NO:3), or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

### II. Glp-1/Glucagon Agonist Peptides

Provided herein are peptides which bind both to a glucagon receptor and to a GLP-1 receptor. Exemplary peptides such as cotadutide (G933; MEDI0382) are provided in WO 2014/091316 and WO 2017/153575, each of which is herein incorporated by reference in its entirety. In some aspects provided herein, the peptide is cotadutide, i.e., a 30 amino acid linear peptide with the sequence of HSQGTFTSDX10SEYLDSERARDFVAWLEAGG-acid, wherein X₁₀=lysine with a palmitoyl group conjugated to the epsilon nitrogen, through a gamma glutamic acid linker (i.e., K(gE-palm)) (SEQ ID NO:4) (see FIG. 1.). In some aspects, the peptides provided herein are co-agonists of glucagon and GLP-1 activity. Such peptides are referred to herein as GLP-1/glucagon agonist peptides. GLP-1/glucagon agonist peptides as provided herein possess GLP-1 and glucagon activities with favorable ratios to promote weight loss, prevent weight gain, or to maintain a desirable body weight, and possess optimized solubility, formulatability, and stability. In some aspects, GLP-1/glucagon agonist peptides as provided herein are active at the human GLP1 and human glucagon receptors. In some aspects, GLP-1/glucagon agonist peptides as disclosed have desirable potencies at the glucagon and GLP-1 receptors, and have desirable relative potencies for promoting weight loss.

Cotadutide has a glutamate residue at position 12, and maintains robust activity at both the glucagon and GLP-1 receptors. The corresponding residue is lysine in exendin-4 (exenatide) and glucagon and is serine in GLP-1. Although this residue is not thought to contact the receptor, changes in charge from positive to negative may modify the adjacent environment. Furthermore, cotadutide has a glutamate residue at position 27. Residue 27 is Lysine in exendin 4 and is an uncharged hydrophobic residue in GLP1 (valine) and glucagon (methionine). The lysine of exendin 4 makes electrostatic interactions with the GLP1 receptor at residues Glu127 and Glu24 (C. R.Underwood et al J Biol Chem 285 723-730 (2010); S.Runge et al J Biol Chem 283 11340-11347 (2008)). While a loss of GLP1R potency might be expected when the charge at position 27 is changed to negative, the change is compatible with GLP1R activity in cotadutide.

Cotadutide is palmitoylated to extend its half-life by association with serum albumin, thus reducing its propensity for renal clearance.

Alternatively or in addition, a GLP-1/glucagon agonist peptide as disclosed herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

Cotadutide can be administered in a titrated dose, e.g., at an initial dose, then at a second higher dose, and optionally at a third higher dose thereafter. The initial dose, and optionally the second dose, can be administered for about 7 days to about 14 days. The initial dose can be at least 20 μg daily. The highest dose (e.g., the second dose or the third dose) can be a dose that does not exceed 600 μg daily. The highest dose (e.g., the second dose or the third dose) can be a dose that does not exceed 300 μg daily.

### III. Methods of Making Glp-1/Glucagon Agonist Peptides

GLP-1/glucagon agonist peptides for uses provided herein can be made by any suitable method. For example, in some aspects provided herein, the GLP-1/glucagon agonist peptides for uses provided herein are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316, which is herein incorporated by reference in its entirety.

Alternatively, GLP-1/glucagon agonist peptides for uses provided herein can be produced recombinantly using a convenient vector/host cell combination as would be well known to the person of ordinary skill in the art. A variety of methods are available for recombinantly producing GLP-1/glucagon agonist peptides. Generally, a polynucleotide sequence encoding the GLP-1/glucagon agonist peptide is inserted into an appropriate expression vehicle, e.g., a vector which contains the necessary elements for the transcription and translation of the inserted coding sequence. The nucleic acid encoding the GLP-1/glucagon agonist peptide is inserted into the vector in proper reading frame. The expression vector is then transfected into a suitable host cell which will express the GLP-1/glucagon agonist peptide. Suitable host cells include without limitation bacteria, yeast, or mammalian cells. A variety of commercially-available host-expression vector systems can be utilized to express the GLP-1/glucagon agonist peptides described herein.

IV. DAPAGLIFLOZIN

Dapagliflozin is a sodium-glucose cotransport 2 (SGLT2) inhibitor. It is described chemically as D-glucitol, 1,5-anhydro-1-C-[4-chloro-3-[(4ethoxyphenyOmethyl]phenyl]—, (1S)—, compounded with (2S)-1,2-propanediol, hydrate (1:1:1). Dapagliflozin has been approved for use in improving glycemic control in adults with T2DM as an adjunct to diet and exercise.

Dapagliflozin is available commercially is Farxiga®. Farxiga® tablets contain 5 mg or 10 mg of dapagliflozin. Farxiga® tablets contain the following inactive ingredients: microcrystalline cellulose, anhydrous lactose, crospovidone, silicon dioxide, and magnesium stearate. Farxiga® tablets also contain a film-coating that contains polyvinyl alcohol, titanium dioxide, polyethylene glycol, talc, and yellow iron oxide.

Dapagliflozin can be administered orally, e.g., as an oral tablet. Dapagliflozin can be administered in the morning, with or without food.

### V. Metformin

As used herein, the terms “metformin” and “metformin hydrochloride” are used interchangeably to refer to N,N-dimethylimidodicarbonimidic diamide hydrochloride. Metformin has been approved for use in the management of T2DM.

Metformin is available commercially as Glucophage® Tablets and as Glucophage® XR Extended-Release Tablets. Glucophage® Tablets contain 500 mg, 850 mg, or 1000 mg of metformin. Each tablet contains the inactive ingredients povidone and magnesium stearate. In addition, the coating for the 500 mg and 850 mg tablets contains hypromellose, and the coating for the 1000 mg tablet contains hypromellose and polyethylene glycol. Glucophage® XR Extended-Release Tablets contain 500 mg or 750 mg of metformin. Glucophage® XR 500 mg tablets contain the inactive ingredients sodium carboxymethyl cellulose, hypromellose, microcrystalline cellulose, and magnesium stearate. Glucophage® XR 750 mg tablets contain the inactive ingredients sodium carboxymethyl cellulose, hypromellose, and magnesium stearate.

Metformin can be administered orally, e.g., as an oral tablet.

Metformin can be administered at a dose of 500 mg to 2550 mg daily. Metformin can be administered at a dose of 500 mg to 2000 mg daily. Metformin can be administered at a dose of 500 mg to 1000 mg daily. Metformin can be administered at a dose of 500 mg to 850 mg daily.

### VI. Pharmaceutical Compositions and Kits

As provided herein, GLP-1/glucagon agonist peptides (e.g., cotadutide) can be used in combination with dapagliflozin and metformin to improve glycemic control, reduce body weight, and/or treat T2DM in a human patient in need thereof.

The GLP-1/glucagon agonist peptides (e.g., cotadutide), the dapagliflozin, and the metformin can each be administered in a separate pharmaceutical composition.

In some aspects, a kit is provided with a GLP-1/glucagon agonist peptide (e.g., cotadutide), dapagliflozin, and metformin. In certain aspects, a kit comprises a GLP-1/glucagon agonist peptide (e.g., cotadutide) and instructions to administer the GLP-1/glucagon agonist peptide (e.g., cotadutide) with dapagliflozin and metformin. In some aspects, a kit comprises a GLP-1/glucagon agonist peptide (e.g., cotadutide), dapagliflozin, and instructions to administer the GLP-1/glucagon agonist peptide (e.g., cotadutide) and dapagliflozin with metformin. In some aspects, a kit comprises a GLP-1/glucagon agonist peptide (e.g., cotadutide), metformin, and instructions to administer the GLP-1/glucagon agonist peptide (e.g., cotadutide) and metformin with dapagliflozin. In some aspects, a kit comprises dapagliflozin, metformin, and instructions to administer the dapagliflozin and metformin with a GLP-1/glucagon agonist peptide (e.g., cotadutide).

A pharmaceutical composition comprising a GLP-1/glucagon agonist peptide (e.g., cotadutide) can be formulated for injection. A pharmaceutical composition comprising a GLP-1/glucagon agonist peptide (e.g., cotadutide) can be formulated for subcutaneous administration.

A pharmaceutical composition comprising a GLP-1/glucagon agonist peptide (e.g., cotadutide) can comprise about 100 mg, about 200 mg, or about 300 mg of the GLP-1/glucagon agonist peptide (e.g., cotadutide).

A pharmaceutical composition comprising dapagliflozin can be formulated for oral administration. A pharmaceutical composition comprising dapagliflozin can be a tablet for oral administration.

A pharmaceutical composition comprising dapagliflozin can comprise about 5 mg or about 10 mg of dapagliflozin.

A pharmaceutical composition comprising metformin can be formulated for oral administration. A pharmaceutical composition comprising metformin can be a tablet for oral administration.

A pharmaceutical composition comprising metformin can comprise about 500 mg, about 750 mg, about 850 mg, or about 1000 mg of metformin.

### VII. Methods of Use

As provided herein, GLP-1/glucagon agonist peptides (e.g., cotadutide) can be used in combination with dapagliflozin and metformin to improving glycemic control, reduce body weight, and/or treat T2DM in a human patient in need thereof.

As provided herein a method of improving glycemic control in a human subject in need thereof can comprise administering to the subject a GLP-1/glucagon agonist peptide (e.g., cotadutide), dapagliflozin, and metformin. This disclosure also provides a GLP-1/glucagon agonist peptide (e.g., cotadutide), dapagliflozin, and metformin (optionally in separate pharmaceutical compositions) for improving glycemic control in a human subject in need thereof. This disclosure also provides a GLP-1/glucagon agonist peptide (e.g., cotadutide), dapagliflozin, and metformin (optionally in separate pharmaceutical compositions) for use in the manufacture of a medicament for improving glycemic control in a human subject in need thereof. The GLP-1/glucagon agonist peptide (e.g., cotadutide) for improving glycemic control can be administered or for administration at a dose of 20-600 μg or 100-300 μg, optionally wherein the administration is by injection (e.g., subcutaneous administration). The GLP-1/glucagon agonist peptide (e.g., cotadutide) for improving glycemic control can be administered or for administration daily (e.g., at daily doses of 20-600 μg or 100-300 μg), optionally wherein the administration is by injection (e.g., subcutaneous administration). The GLP-1/glucagon agonist peptide (e.g., cotadutide) for improving glycemic control can be administered or for administration in titrated doses, e.g., at an initial dose of 100 μg, then a second dose of 200 μg, then a third dose of 300 μg. The initial dose can be administered for about 7 days. The second dose can be administered for about 7 days. The dapagliflozin for improving glycemic control can be administered or for administration at a dose of 10 mg, optionally wherein the administration is oral (e.g. by oral tablet). The metformin for improving glycemic control can be administered or for administration at a dose of e.g., 500 mg to 2550 mg daily, optionally wherein the administration is oral (e.g. by oral tablet). The administration can be an adjunct to diet and exercise.

As provided herein a method of reducing weight in a human subject in need thereof can comprise administering to the subject a GLP-1/glucagon agonist peptide (e.g., cotadutide), dapagliflozin, and metformin. This disclosure also provides a GLP-1/glucagon agonist peptide (e.g., cotadutide), dapagliflozin, and metformin (optionally in separate pharmaceutical compositions) for reducing weight in a human subject in need thereof. This disclosure also provides a GLP-1/glucagon agonist peptide (e.g., cotadutide), dapagliflozin, and metformin (optionally in separate pharmaceutical compositions) for use in the manufacture of a medicament for reducing weight in a human subject in need thereof. The GLP-1/glucagon agonist peptide (e.g., cotadutide) for reducing weight can be administered or for administration at a dose of 20-600 μg or 100-300 optionally wherein the administration is by injection (e.g., subcutaneous administration). The GLP-1/glucagon agonist peptide (e.g., cotadutide) for reducing weight can be administered or for administration daily (e.g., at daily doses of 20-600 μg or 100-300 μg), optionally wherein the administration is by injection (e.g., subcutaneous administration). The GLP-1/glucagon agonist peptide (e.g., cotadutide) for reducing weight can be administered or for administration in titrated doses, e.g., at an initial dose of 100 μg, then a second dose of 200 then a third dose of 300 μg. The initial dose can be administered for about 7 days. The second dose can be administered for about 7 days. The dapagliflozin for reducing weight can be administered or for administration at a dose of 10 mg, optionally wherein the administration is oral (e.g. by oral tablet). The metformin for reducing weight can be administered or for administration at a dose of e.g., 500 mg to 2550 mg daily, optionally wherein the administration is oral (e.g. by oral tablet). The administration can be an adjunct to diet and exercise.

As provided herein a method of treating T2DM in a human subject in need thereof can comprise administering to the subject a GLP-1/glucagon agonist peptide (e.g., cotadutide), dapagliflozin, and metformin. This disclosure also provides a GLP-1/glucagon agonist peptide (e.g., cotadutide), dapagliflozin, and metformin (optionally in separate pharmaceutical compositions) for treating T2DM in a human subject in need thereof. This disclosure also provides a GLP-1/glucagon agonist peptide (e.g., cotadutide), dapagliflozin, and metformin (optionally in separate pharmaceutical compositions) for use in the manufacture of a medicament for treating T2DM in a human subject in need thereof. The GLP-1/glucagon agonist peptide (e.g., cotadutide) for treating T2DM can be administered or for administration at a dose of 20-600 μg or 100-300 optionally wherein the administration is by injection (e.g., subcutaneous administration). The GLP-1/glucagon agonist peptide (e.g., cotadutide) for treating T2DM can be administered or for administration daily (e.g., at daily doses of 20-600 μg or 100-300 μg), optionally wherein the administration is by injection (e.g., subcutaneous administration). The GLP-1/glucagon agonist peptide (e.g., cotadutide) for treating T2DM can be administered or for administration in titrated doses, e.g., at an initial dose of 100 μg, then a second dose of 200 then a third dose of 300 μg. The initial dose can be administered for about 7 days. The second dose can be administered for about 7 days. The dapagliflozin for treating T2DM can be administered or for administration at a dose of 10 mg, optionally wherein the administration is oral (e.g. by oral tablet). The metformin for treating T2DM can be administered or for administration at a dose of e.g., 500 mg to 2550 mg daily, optionally wherein the administration is oral (e.g. by oral tablet). The administration can be an adjunct to diet and exercise.

In some aspects provided herein, the GLP-1/glucagon agonist peptide (e.g., cotadutide), dapagliflozin, and metformin can reduce the mixed-meal tolerance test (MMTT) plasma glucose area under the curve (AUC)₀₋₄ₕₒᵤᵣₛ in a patient. In some aspects, the MMTT plasma glucose AUC₀₋₂₄ hours can be reduced by at least 25 mg-hr/dL, at least 50 mg-hr/dL, at least 75 mg-hr/dL, at least 100 mg-hr/dL, or at least 150 mg-hr/dL. In some aspects, the MMTT plasma glucose AUC₀₋₂₄ hours can be reduced 25-200 mg-hr/dL, 50-200 mg-hr/dL, 75-200 mg-hr/dL, 100-200 mg-hr/dL, or 150-200 mg-hr/dL. In some aspects, the percent MMTT plasma glucose AUC₀₋₂₄ hours can be reduced by at least 5%, at least 10%, at least 15%, or at least 20%. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce continuous glucose monitoring (CGM) glucose AUC₀₋₂₄ in a patient. In some aspects, the CGM glucose AUC₀₋₂₄ can be reduced by at least 200 mg-hr/dL, at least 250 mg-hr/dL, at least 300 mg-hr/dL, at least 350 mg-hr/dL, at least 400 mg-hr/dL, at least 450 mg-hr/dL, at least 500 mg-hr/dL, at least 550 mg-hr/dL, at least 600 mg-hr/dL, or at least 650 mg-hr/dL. In some aspects, the CGM glucose AUC₀₋₂₄ can be reduced by 200-750 mg-hr/dL, 250-750 mg-hr/dL, 300-750 mg-hr/dL, 350-750 mg-hr/dL, 400-750 mg-hr/dL, 450-750 mg-hr/dL, 500-750 mg-hr/dL, 550-750 mg-hr/dL, 600-750 mg-hr/dL, or 650-750 mg-hr/dL. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce 24-hour CGM mean glucose in a patient. In some aspects, the 24-hour CGM mean glucose can be reduced by at least 10 mg/dL, at least 15 mg/dL, at least 20 mg/dL, or at least 25 mg/dL. In some aspects, the 24-hour CGM mean glucose can be reduced by 10-35 mg/dL, 15-35 mg/dL, 20-35 mg/dL, or 25-35 mg/dL. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce the standard deviation (SD) in CGM glucose in a patient by at least 5 mg/dL. In some aspects provided herein, the GLP-1/glucagon agonist peptide (e.g., cotadutide), dapagliflozin, and metformin can reduce the standard deviation (SD) in CGM glucose in a patient by 5-15 mg/dL. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce the CGM mean amplitude of glucose excursion (MAGE) in a patient by at least 10 mg/dL, at least 15 mg/dL, at least 20 mg/dL, or at least 25 mg/dL. In some aspects provided herein, the GLP-1/glucagon agonist peptide (e.g., cotadutide), dapagliflozin, and metformin can reduce the CGM MAGE in a patient by 10-35 mg/dL, 15-35 mg/dL, 20-35 mg/dL, or 25-35 mg/dL. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce fasting plasma glucose (FPG) in a patient. In some aspects, the FPG can be reduced by at least 5 mg/dL, at least 10 mg/dL, at least 15 mg/dL, at least 20 mg/dL, at least 25 mg/dL, or at least 30 mg/dL. In some aspects, the FPG can be reduced by 5-50 mg/dL, 10-50 mg/dL, 15-50 mg/dL, 20-50 mg/dL, 25-50 mg/dL, or 30-50 mg/dL. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce Hemoglobin A1c (HbA1c) in a patient by at least 0.5% or by at least 1%. In some aspects provided herein, the GLP-1/glucagon agonist peptide (e.g., cotadutide), dapagliflozin, and metformin can reduce HbA1c in a patient by 0.5-2% or by 1-2%. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can reduce body weight of a patient by at least 2 kg or by at least 3 kg. In some aspects provided herein, the GLP-1/glucagon agonist peptide (e.g., cotadutide), dapagliflozin, and metformin can reduce body weight of a patient 2-10 kg or by 3-10 kg. The reduction can occur, e.g., within 28 days from the first administration of the GLP-1/glucagon agonist peptide (e.g., cotadutide).

In some aspects provided herein, the GLP-1/glucagon agonist peptide (e.g., cotadutide), dapagliflozin, and metformin can produce euglycemic glucose levels in a patient. In some aspects provided herein, the GLP-1/glucagon agonist peptide (e.g., cotadutide), dapagliflozin, and metformin can prevent hyperglycemic glucose levels in the patient.

As provided herein, the human subject discussed in any of the aspects above can have type 2 diabetes mellitus.

As provided herein, the human subject discussed in any of the aspects above can have a body mass index (BMI) of 25 to 40 kg/m².

As provided herein, the human subject discussed in any of the aspects above can have an hemoglobin A1c (HbA1c) of >7.0% to <10.0%.

In some aspects, a human subject provided herein is receiving treatment with metformin prior to the administration of the combination of a GLP-1/glucagon agonist peptide (e.g., cotadutide), dapagliflozin, and metformin. In some aspects, a human subject provided herein is receiving treatment with metformin MTD>1 g prior to the administration of the combination of a GLP-1/glucagon agonist peptide (e.g., cotadutide), dapagliflozin, and metformin.

In some aspects, a human subject provided herein is receiving treatment with dapagliflozin and metformin prior to the administration of the combination of a GLP-1/glucagon agonist peptide (e.g., cotadutide), dapagliflozin, and metformin. In some aspects, a human subject provided herein is receiving treatment with 10 mg dapagliflozin and metformin MTD>1 g prior to the administration of the combination of a GLP-1/glucagon agonist peptide (e.g., cotadutide), dapagliflozin, and metformin.

### EXAMPLES

### Example 1: Cotadutide with Dapagliflozin and Metformin Dual Therapy

A Phase 2a randomized, placebo-controlled, double-bind study was performed to demonstrate the efficacy and safety of cotadutide in overweight/obese subjects with Type 2 Diabetes Mellitus (T2DM) treated with dapagliflozin and metformin dual therapy.

### (A) Subjects

A total of 128 subjects consented to participate in the study. The study enrolled subjects with T2DM treated either with metformin monotherapy or with metformin and dapagliflozin dual therapy. The subjects were screened for the following inclusion and exclusion criteria.

Inclusion criteria:
Male and female subjects, aged≥18 years at screening;
Body mass index (BMI) between 25 kg/m² and 40 kg/m² (inclusive) at screening;
HbA1c range between 7.0% and 10.0% (inclusive) at the time of screening;
Diagnosed with T2DM and treated with of metformin monotherapy (MTD>1 g) at least 8 weeks prior to screening or treated with stable, oral doses of dapagliflozin 10 mg and metformin (MTD>1 g) for at least 3 months prior screening. Exclusion criteria:
Any subject who had received any of the following medications prior to the start of the screening period (Visit 1) or prior to the study start period (Visit 4):
   Concurrent use of any medicinal products, or herbal or over-the-counter (OTC) preparations licensed for control of body weight or appetite at the time of screening (Visit 1);
   Concurrent or previous use of drugs approved for weight loss (e.g., orlistat, bupropion-naltrexone, phentermine-topiramate, phentermine, lorcaserin) within the last 30 days or 5 half-lives of the drug (whichever was longest) at the time of screening (Visit 1);
   Concurrent use of aspirin (acetylsalicylic acid) at a dose greater than 150 mg once daily and within the last 72 hours prior to the start of the study (Visit 4);
   Concurrent use of paracetamol (acetaminophen) or paracetamol-containing preparations at a total daily dose of greater than 3000 mg and within the last 72 hours prior to the start of the study (Visit 4);
   Concurrent use of ascorbic acid (vitamin C) supplements at a total daily dose greater than 1000 mg and within the last 72 hours prior to the start of the study (Visit 4); or
   Concurrent use of opiates, domperidone, metoclopramide, or other drugs known to alter gastric emptying and within the last 72 hours prior to the start of the study (Visit 4);
Diagnosis of type 1 diabetes mellitus, maturity-onset diabetes of the young, or latent autoimmune diabetes of adulthood or presence of anti-glutamic acid decarboxylase, anti-islet cell, or anti-insulin antibodies;
Symptoms of acutely decompensate blood glucose control (e.g., thirst, polyuria, weight loss) at screening or randomization, a history of diabetes ketoacidosis (DKA), or hyperosmolar nonketotic coma or treatment with daily subcutaneous (SC) insulin within 90 days prior to screening;
Fasting hyperglycemia (>250 mg/dL>13.9 mmol/L) prior to randomization;
C-peptide level<lower limit of normal;
History of acute or chronic pancreatitis or pancreatectomy;
Hypertriglyceridemia (>400 mg/dL) at screening;
Significant inflammatory bowel disease, gastroparesis, or other severe disease or surgery affecting the upper gastrointestinal tract (including weight-reducing surgery and procedures) which may have affected gastric emptying or could have affected the interpretation of safety and tolerability data;
Significant hepatic disease (except for nonalcoholic steatohepatitis or nonalcoholic fatty liver disease without portal hypertension or cirrhosis) and/or subjects with any of the following results at screening: aspartate transaminase (AST)≥3× upper limit of normal (ULN), alanine transaminase (ALT)≥3×ULN, or total bilirubin (TBL)≥2×ULN;
Impaired renal function defined as estimated glomerular filtration rate (eGFR)≤60 mL/minute/1.73 m² at screening (eGFR according to Modification of Diet in Renal Disease [MDRD] using the isotope dilution mass spectrometry-traceable MDRD Study Equation (International system of units [SI]);
Use of loop diuretics within 1 month prior to screening;
Poorly controlled hypertension defined as follows: Systolic BP>160 mmHg, Diastolic BP or >100 mmHg after 10 minutes of supine rest and confirmed by repeated measurement at screening (Visit 1 for all subjects);
Unstable angina pectoris, myocardial infarction, transient ischemic attack, or stroke within 3 months prior to screening, or subjects who have undergone percutaneous coronary intervention or a coronary artery bypass graft within the past 6 months or who were due to undergo these procedures at the time of screening;
Severe congestive heart failure (New York Heart Association Class III and IV);
Basal calcitonin level>50 ng/L at screening or history/family history of medullary thyroid carcinoma or multiple endocrine neoplasia;
Hemoglobinopathy, hemolytic anemia, or chronic anemia (hemoglobin concentration<11.5 g/dL (115 g/L) for males and <10.5 g/dL (105 g/L) for females) at screening or any other condition known to interfere with interpretation of HbA1c measurement;
History of neoplastic disease within 5 years prior to screening, except for adequately treated basal cell skin cancer, squamous cell skin cancer, or in situ cervical cancer;
Any positive results for serum hepatitis B surface antigen, hepatitis C antibody, and human immunodeficiency virus antibody;
Recent viral infection or illness requiring the use of antibiotics in the month prior to screening (Visit 1) for subjects on dual therapy or prior to run—in period (Visit 2) for subjects on monotherapy; and
History of recurrent (at least 2) urinary tract and/or genital tract infections (including mycotic infections such as thrush) within 6 months prior to screening.

Of the 128 subjects who consented to participate, 79 subjects were considered screen failures, and 49 subjects were randomized (25 subjects to the cotadutide group and 24 subjects to the placebo group).

A total of 47 subjects (95.9%) completed the study, and a total of 46 subjects (93.9%) completed treatment as planned. Three subjects (6.1%, all in the cotadutide treatment groups) discontinued treatment: 2 subjects (4.1%) due to an adverse event (AE), and 1 subject (2.0%) discontinued due to withdrawal by the subject.

All 49 subjects who received investigational product were included in the Intent-to-treat (ITT), As-treated, and Immunogenicity populations. A total of 25 subjects who received cotadutide had evaluable post-dose PK data and were included in the cotadutide PK population. A total of 49 subjects were included in the Dapagliflozin population, and 48 subjects were included in the Ketone PK populations. The analysis populations were grouped as follows:
Intent-to-treat (ITT) Population=Randomized subjects who received any investigational product (cotadutide or placebo) analyzed according to their randomized treatment group.
As-treated Population=Subjects who received any investigation product (cotadutide or placebo) analyzed according to the treatment they actually received.
cotadutide pharmacokinetic (PK) Population=Subjects who received at least 1 dose of investigational product (cotadutide or placebo) and had at least 1 cotadutide PK sample taken that was above the lower limit of quantitation.
Dapagliflozin PK Population=Subjects who received at least 1 dose of investigational product (dapagliflozin or placebo) and had at least 1 dapagliflozin PK sample for dapagliflozin taken that was above the lower limit of quantitation.
Ketone PK Population=Subjects who received at least 1 dose of investigational product (cotadutide or dapagliflozin or placebo) and had at least 1 ketone PK sample taken that was above the lower limit of quantitation.
Immunogenicity Population=Subjects in the As-treated population (cotadutide and placebo) who had at least one serum sample for immunogenicity testing.

The analysis populations are summarized in Table 1. No subjects were excluded from any of the analysis populations.

TABLE 1
Analysis Populations

| Population | Placebo | Cotadutide | Total |
|---|---|---|---|
| ITT population | 24 | 25 | 49 |
| As-treated population | 24 | 25 | 49 |
| Immunogenicity population | 24 | 25 | 49 |
| Cotadutide PK population | 0 | 25 | 25 |
| Dapagliflozin PK population | 24 | 25 | 49 |
| Ketone PK population | 24 | 24 | 48 |

Demographic and baseline disease characteristics were generally balanced across treatment groups. (See Table 2.) The mean age of the As-treated Population was 59.7 years (range: 41 to 74 years) with the majority of subjects being male. The mean weight was 95.86 kg, with subjects in the cotadutide group weighing less than subjects in the placebo group at baseline (92.24 vs 99.63 kg, respectively); the mean total BMI was 33.279 kg/m² and was similar between groups. The mean height was 169.43 cm. The mean duration of T2DM was 8.28 years, with longer duration of disease in the cotadutide group compared with the placebo group (9.16 vs 7.36 years, respectively). The mean HbA1c at baseline was 7.69% overall (range: 6.5 to 10.1%), and over half of subjects were on metformin monotherapy at baseline (57.1%).

TABLE 2
Subject Demographics and Baseline Disease Characteristics (As-treated Population)

| | | | |
|---|---|---|---|
| | Placebo | Cotadutide | Total |
| Parameter | N = 24 | N = 25 | N = 49 |

Age (years)

| n | 24 | | 25 | | 49 | |
|---|---|---|---|---|---|---|
| Mean (SD) | 58.4 | (10.0) | 61.0 | (8.2) | 59.7 | (9.1) |
| Median (Min, Max) | 60.0 | (41, 73) | 63.0 | (42, 74) | 63.0 | (41, 74) |

Sex

| n | 24 | | 25 | | 49 | |
|---|---|---|---|---|---|---|
| Female | 12 | (50.0%) | 10 | (40.0%) | 22 | (44.9%) |
| Male | 12 | (50.0%) | 15 | (60.0%) | 27 | (55.1%) |

Weight (kg)

| n | 24 | | 25 | | 49 | |
|---|---|---|---|---|---|---|
| Mean (SD) | 99.63 | (16.87) | 92.24 | (14.19) | 95.86 | (15.84) |
| Median (Min, Max) | 95.00 | (74.3, 134.9) | 89.60 | (63.0, 127.6) | 93.10 | (63.0, 134.9) |

Height (cm)

| n | 24 | | 25 | | 49 | |
|---|---|---|---|---|---|---|
| Mean (SD) | 171.39 | (11.75) | 167.56 | (8.34) | 169.43 | (10.23) |
| Median (Min, Max) | 172.00 | (148.0, 192.0) | 170.00 | (151.0, 183.0) | 171.00 | (148.0, 192.0) |

Body mass index (kg/m²)

| n | 24 | | 25 | | 49 | |
|---|---|---|---|---|---|---|
| Mean (SD) | 33.838 | (3.867) | 32.743 | (3.473) | 33.279 | (3.674) |
| Median (Min, Max) | 34.450 | (27.46, 40.36) | 33.070 | (26.22, 39.82) | 33.610 | (26.22, 40.36) |

Duration of Type 2 Diabetes (years)

| n | 24 | | 25 | | 49 | |
|---|---|---|---|---|---|---|
| Mean (SD) | 7.36 | (6.10) | 9.16 | (5.69) | 8.28 | (5.90) |
| Median (Min, Max) | 6.90 | (0.3, 28.0) | 9.00 | (1.4, 20.0) | 7.00 | (0.3, 28.0) |

HbA1c (%)

| n | 24 | | 25 | | 49 | |
|---|---|---|---|---|---|---|
| Mean (SD) | 7.60 | (0.80) | 7.78 | (0.83) | 7.69 | (0.81) |
| Median (Min, Max) | 7.45 | (6.5, 9.9) | 7.60 | (6.5, 10.1) | 7.60 | (6.5, 10.1) |

Metformin monotherapy

| | | | | | | |
|---|---|---|---|---|---|---|
| Yes | 13 | (54.2%) | 15 | (60.0%) | 28 | (57.1%) |
| No | 11 | (45.8%) | 10 | (40.0%) | 21 | (42.9%) |

^{a} HbA1c = glycated hemoglobin; Max = maximum; Min = minimum; SD = standard deviation
^{b} Note:
Subjects were on metformin and dapagliflozin background dual therapy during the double-blind treatment period.

### (B) Study Design

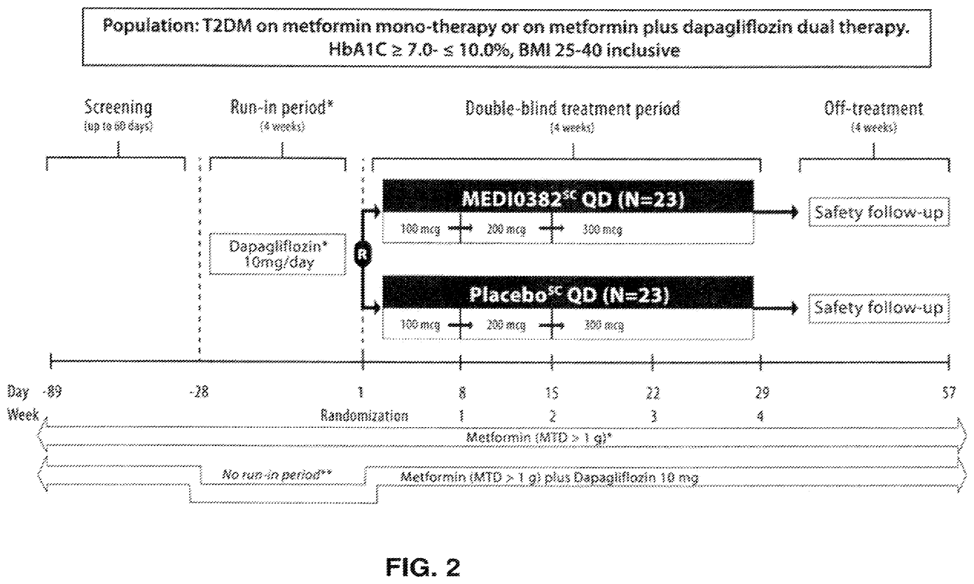

A flow diagram of the proposed study is provided in FIG. 2. After the screening period, subjects treated with metformin monotherapy only entered a 4-week run—in period, where subjects were administered oral dapagliflozin 10 mg a day. Enrolled subjects that were already treated with metformin and dapagliflozin dual therapy continued this dual therapy (10 mg dapagliflozin and metformin (maximum tolerated dose (MTD) of >1 g)) throughout the study.

Following the run—in period for subjects on metformin monotherapy with a fasting plasma glucose (FPG)>7.0 mmol/L (126 mg/dL) or the screening period for subjects on metformin and dapagliflozin dual therapy, subjects were randomized to the following treatment groups in the 4-week treatment period:
Cotadutide subcutaneously (SC) once daily (titrated up from 100 μg for 7 days to 200 μg for 7 days and to 300 μg for 14 days) in the morning for 28 days (N=23); or
Placebo SC once daily in the morning for 28 days (N=23).

The first day of dosing with cotadutide or placebo was considered Day 1. On the day of each dose, investigational product (cotadutide or placebo) was administered as noted above. On study days where fasting was required for an additional assessment, the investigational product was taken approximately 2.5 hours prior to the applicable assessment. For at-home treatment administration, a once-daily dose was to be self-administered by SC injection using the prefilled syringe as soon as practicable upon waking each morning prior to breakfast.

### Mixed Meal Tolerance Test

Subjects were admitted to the unit on Day −2 and on Day 27. On Day −1 and on Day 28, following a minimum 10 hour fast, a blood sample for glucose metabolism panel (glucose, insulin, C-peptide, glucagon, and active GLP-1) was taken immediately prior to the subject drinking a standardized mixed meal (t=“0 minutes”). The subject then consumed a can of Ensure Plus, which is a nutritional supplement containing the components of fat, carbohydrate, and protein that make up a standard mixed-meal tolerance test (MMTT). After the meal, timed, serial blood samples were obtained for measurement of glucose and parameters related to glucose metabolism (with no additional food intake during this time). The MMTT was performed on Day-1 and Day 28.

Blood samples for the glucose metabolism panel were drawn at t=“0 minutes” (before meal) and at 15, 30, 45, 60, 90, 120, 180, and 240 minutes (±5 minutes) after consumption of the standardized meal. Additional sampling for glucagon, insulin, C-peptide, and glucose was taken at 6, 8, 12, and 24 hours (±30 minutes) post-administration (t=“0 minutes”) of the standardized meal on Day −1 and 28.

The change and percent change from baseline (Day-1) to the end of the 28 days of treatment (Day 28) in glucose AUC₀₋₄ h were determined as measured by the MMTT. In addition, the change from baseline (Day −1) to Day 28 in active GLP-1, glucagon, insulin, and C-peptide AUC₀₋₄ h were also measured by MMTT.

### Continuous Glucose Monitoring

A continuous glucose monitoring (CGM) device was used to measure interstitial glucose levels during the study. Subjects wore the CGM sensor continuously up until the time of a sensor change (every 7 to 14 days). If a subject was unable to tolerate wearing the CGM sensor for the entire duration of the study, the sensor was to be removed; but the subject should have remained in the study with or without continued CGM.

### Glucometer Measure Capillary Blood Glucose/Ketones Reading

At the start of the study, each subject in both treatment groups was issued a standardized glucometer, testing strips for glucose and ketones, and a diary. For the duration of the study, subjects were encouraged to perform finger-prick tests if they felt unwell and in particular if they felt the symptoms may have been due to hypoglycemia, but they were not required to test routinely. If the investigator or site staff felt that a subject could be experiencing hypo- or hyperglycemia, capillary blood glucose was tested with a standardized glucometer. Capillary blood glucose levels of <3 mmol/L (54 mg/dL) were as an adverse event (AE) regardless of whether the subject had symptoms or not.

During the course of the study, subjects tested their blood ketone level daily (prebreakfast) using the ketone finger-prick testing strips and ketone monitor provided. Subjects were asked to record ketone values in a paper diary, which was provided and checked by study staff during the outpatient visits (i.e., when subjects brought the paper diary back in house). The clinic staff provided training on how to use the ketones monitor.

### Body Weight

Body weight was measured during the screening period, at the start of the run—in period (Day −28), and prior to breakfast on Day 1 (predose) and Day 29. The subject should have taken off their shoes and remove bulky clothing. Calibrated scales were used.

The change and percent change in body weight from baseline (Day 1) to Day 29 (kg) were determined. The proportion of subjects achieving>5% body weight loss from baseline (Day 1) to Day 29 was also determined.

### Pharmacokinetic Evaluations

Blood was collected to evaluate the pharmacokinetics (PK) of cotadutide and dapagliflozin in plasma. The PK of cotadutide and dapagliflozin in plasma was measured utilizing separate validated liquid chromatography-tandem mass spectrometry methods. Blood samples for PK (cotadutide and dapagliflozin) were drawn as follows:

Cotadutide:
Day 7, 14, and 28: Pre-dose; 0.5, 1, and 2 hours (±15 minutes) post-dose; and 4, 6, 8, and 12, (±30 minutes) post-dose;
Day 8 and 15: Pre-dose; and
Day 29: 24 hours after dosing time on Day 28

Dapagliflozin:
Day −1, 7, 14, 28: Pre-dose; 0.5, 1, and 2 hours (±15 minutes) post-dose; and 4, 6, 8, and 12 hours (±30 minutes) post-dose;
Day 1, 8, and 15: Pre-dose; and
Day 29: 24 hours after dosing time on Day 28

### Immunological Evaluations and Adverse Events

Blood samples were collected to evaluate anti-drug antibody (ADA) response to cotadutide. Serum samples were evaluated for ADA using a validated immunoassay. Adverse events (AEs) and serious adverse events (SAEs) were collected from the time of informed consent throughout the treatment period and including the follow-up period. AEs and SAEs were graded by severity and relationship to investigational product and SAEs were assessed for relationship to protocol procedures.

### (C) RESULTS

Clinically meaningful and statistically significantly results were observed for the cotadutide group compared with the placebo group.

### Change in MMTT Plasma Glucose AUC₀₋₂₄h

The change in the mixed-meal tolerance test (MMTT) plasma glucose AUC₀₋₄ h from the baseline evaluation to the Day 28 evaluation was assessed. A statistically significantly greater reduction from baseline to Day 28 in MMTT plasma glucose AUC₀₋₄ h was observed for the cotadutide group compared with the placebo group (LS mean difference: −143.09 mg-hr/dL; p<0.0001) (Table 3).

The percent change in the MMTT plasma glucose AUC₀₋₄ h from the baseline evaluation to the Day 28 evaluation was also assessed. A statistically significantly greater reduction from baseline to Day 28 in MMTT plasma glucose AUC₀₋₄ h was observed for the cotadutide group compared with the placebo group (LS mean difference: −22.17%; p<0.0001) (Table 3).

TABLE 3
Change and Percent Change from Baseline to Day 28 in
Plasma Glucose AUC_{0-4 h} After MMTT (ITT Population)

| | | |
|---|---|---|
| | Placebo | Cotadutide |
| Glucose | N = 24 | N = 25 |

Baseline (mg-hr/dL)

| n | 23 | | 25 | |
|---|---|---|---|---|
| Mean (SD) | 680.43 | (172.64) | 680.72 | (118.53) |
| Median (Min, | 640.45 | (441.5, 1216.8) | 659.76 | (497.1, 1004.5) |
| Max) | | | | |

Day 28 (mg-hr/dL)

| n | 23 | | 24 | |
|---|---|---|---|---|
| Mean (SD) | 673.92 | (121.76) | 523.72 | (128.32) |
| Median (Min, | 645.69 | (415.8, 949.6)) | 481.57 | (361.5, 863.3) |
| Max) | | | | |

Change from baseline

| n | 22 | | 24 | |
|---|---|---|---|---|
| Mean (SD) | −14.64 | (106.07) | −151.29 | (109.16) |
| Median (Min, | −18.47 | (−267.1, 189.8) | −148.29 | (−383.6, 85.5) |
| Max) | | | | |

| | | |
|---|---|---|
| LS mean ^{a} | −11.28 | −154.37 |
| 95% CI of LS | −51.05, 28.50 | −192.45, −116.29 |
| mean ^{a} | | |
| LS mean | — | −143.09 |
| difference ^{a} | | |
| 95% CI ^{a} | — | −198.20, −87.98 |
| p-value ^{a} | — | <0.0001 |

Percent change from baseline

| n | 22 | | 24 | |
|---|---|---|---|---|
| Mean (SD) | −0.43 | (13.33) | −22.03 | (15.11) |
| Median (Min, | −2.74 | (−22.0, 29.5) | −25.55 | (−51.5, 15.5) |
| Max) | | | | |

| | | |
|---|---|---|
| LS mean ^{a} | −0.13 | −22.30 |
| 95% CI of LS | −5.96, 5.69 | −27.88, −16.73 |
| mean ^{a} | | |
| LS mean | — | −22.17 |
| difference ^{a} | | |
| 95% CI ^{a} | — | −30.24, −14.10 |
| p-value ^{a} | — | <0.0001 |

ANCOVA = analysis of covariance;
AUC_{0-4 h} = area under the concentration-time curve from time 0 to 4 hours;
CI = confidence interval;
ITT = intent to treat;
LS = least squares;
Max = maximum;
Min = minimum;
MMTT = mixed-meal tolerance test;
SD = standard deviation
Note:
Subjects were on metformin and dapagliflozin background dual therapy during the double-blind treatment period.
^{a} LS mean, its associated 95% CI, and p-value from ANCOVA with treatment effect and baseline value as covariate.

24-hour Glucose Control as Measured by CGM

Subjects underwent continuous glucose monitoring (CGM) for a total of 28 days with sensor changes every 7 to 14 days. The changes in CGM glucose AUC₀₋₂₄ h and mean glucose over 24 hours from baseline to the end of each dosing level on Days 7 (100 μg), 14 (200 μg), and 28 (300 μg) were measured. Variability in glucose levels was also assessed by measuring the changes in standard deviation (SD), coefficient of variation (CV), and mean amplitude of glucose excursion (MAGE) of CGM glucose values over 24 hours.

Numerically greater mean reductions from baseline in CGM glucose AUC₀₋₂₄h were observed for the cotadutide group compared with the placebo group at Day 7 (−832.66 and 49.14 mg-hr/dL, respectively) and Day 14 (−666.05 and 57.30 mg-hr/dL, respectively). On Day 28, a statistically significantly greater reduction from baseline in CGM glucose AUC₀₋₂₄ h was observed for the cotadutide group compared with the placebo group (LS mean difference: −806.61 mg-hr/dL; p=0.0001) (Table 4).

TABLE 4
Change in CGM Glucose AUC0-24 h from Baseline to
the End of Each Dosing Level (ITT Population)

| | | |
|---|---|---|
| Glucose | Placebo | Cotadutide |
| (mg-hr/dL) | N = 24 | N = 25 |

Baseline

| n | 16 | | 22 | |
|---|---|---|---|---|
| Mean (SD) | 3104.97 | (612.09) | 3228.24 | (720.04) |
| Median (Min, | 3140.44 | (2271.2, 4738.1) | 3211.16 | (2006.1, 5513.7) |
| Max) | | | | |

Day 7

| n | 10 | | 12 | |
|---|---|---|---|---|
| Mean (SD) | 49.14 | (667.30) | −832.66 | (506.22) |
| Median (Min, | −58.00 | (−1095.8, 1188.9) | −962.95 | (−1574.6, 121.4) |
| Max) | | | | |

Day 14

| n | 9 | | 13 | |
|---|---|---|---|---|
| Mean (SD) | 57.30 | (379.95) | −666.05 | (647.63) |
| Median (Min, | −102.04 | (−260.7, 957.0) | −897.02 | (−1441.5, 628.1) |
| Max) | | | | |

Day 28 (LOCF)

| n | 15 | | 20 | |
|---|---|---|---|---|
| Mean (SD) | 196.66 | (537.98) | −695.16 | (695.70) |
| Median (Min, | 160.30 | (−615.6, 1624.3) | −928.48 | (−1638.5, 1081.9) |
| Max) | | | | |

| | | |
|---|---|---|
| LS mean ^{a} | 147.97 | −658.64 |
| 95% CI of LS | −136.03, 431.97 | −904.33, −412.96 |
| mean ^{a} | | |
| LS mean | — | −806.61 |
| difference ^{a} | | |
| 95% CI ^{a} | — | −1183.55, −429.68 |
| p-value ^{a} | — | 0.0001 |

ANCOVA = analysis of covariance;
AUC_{0-24 h} = area under the concentration-time curve from time 0 to 4 hours;
CGM = continuous glucose monitoring;
CI = confidence interval;
ITT = intent-to-treat;
LOCF = last observation carried forward;
LS = least squares;
Max = maximum;
Min = minimum;
SD = standard deviation
Note:
Subjects were on metformin and dapagliflozin background dual therapy during the double-blind treatment period.
^{a} LS Mean, its associated 95% CI, and p-value from ANCOVA with treatment effect and baseline value as covariate.

Numerically greater mean reductions from baseline in CGM mean glucose over 24 hours were observed for the cotadutide group compared with the placebo group at Day 7 (−34.74 and 2.59 mg/dL, respectively) and Day 14 (−28.34 and 2.83 mg/dL, respectively). On Day 28, a statistically significantly greater reduction from baseline in CGM mean glucose was observed for the cotadutide group compared with the placebo group (LS mean difference: −34.06 mg/dL; p=0.0001).

Numerically greater mean reductions from baseline in SD of CGM glucose over 24 hours were observed for the cotadutide group compared with the placebo group at Day 7 (−7.21 and −3.01 mg/dL, respectively) and Day 14 (−7.52 and 1.38 mg/dL, respectively). On Day 28, a statistically significantly greater reduction from baseline in SD of CGM glucose was observed for the cotadutide group compared with the placebo group (LS mean difference: −8.45 mg/dL; p=0.0029) (Table 5).

TABLE 5
Change in SD over 24 Hours in CGM Glucose from Baseline
to the End of Each Dosing Level (ITT Population)

| | | |
|---|---|---|
| | Placebo | Cotadutide |
| Glucose (mg/dL) | N = 24 | N = 25 |

Baseline

| n | 16 | | 22 | |
|---|---|---|---|---|
| Mean (SD) | 32.52 | (10.07) | 29.52 | (6.80) |
| Median (Min, Max) | 32.76 | (13.6, 47.0) | 30.46 | (16.5, 41.6) |

Day 7

| n | 10 | | 12 | |
|---|---|---|---|---|
| Mean (SD) | −3.01 | (13.84) | −7.21 | (11.05) |
| Median (Min, Max) | −2.95 | (−26.3, 22.5) | −11.16 | (−18.5, 16.0) |

Day 14

| n | 9 | | 13 | |
|---|---|---|---|---|
| Mean (SD) | 1.38 | (9.47) | −7.52 | (9.73) |
| Median (Min, Max) | −0.80 | (−13.5, 16.2) | −8.36 | (−20.3, 14.9) |

Day 28 (LOCF)

| n | 15 | | 20 | |
|---|---|---|---|---|
| Mean (SD) | −2.38 | (10.80) | −8.89 | (10.08) |
| Median (Min, Max) | −0.80 | (−22.8, 15.0) | −9.57 | (−25.9, 16.4) |

| | | |
|---|---|---|
| LS mean ^{a} | −1.27 | −9.73 |
| 95% CI of LS mean ^{a} | −5.29, 2.74 | −13.20, −6.25 |
| LS mean difference ^{a} | — | −8.45 |
| 95% CI ^{a} | — | −13.79, −3.12 |
| p-value ^{a} | — | 0.0029 |

ANCOVA = analysis of covariance;
AUC_{0-24 h} = area under the concentration-time curve from time 0 to 24 hours;
CGM = continuous glucose monitoring;
CI = confidence interval;
ITT = intent-to-treat;
LOCF = last observation carried forward;
LS = least squares;
Max = maximum;
Min = minimum;
SD = standard deviation
Note:
Subjects were on metformin and dapagliflozin background dual therapy during the double-blind treatment period.
^{a} LS mean, its associated CI, and p-value from analysis of covariance adjusted by baseline value.

There were no meaningful differences between groups in CV of CGM glucose over 24 hours from baseline to Day 7, Day 14, or Day 28 (LS mean difference at Day 28: −2.41%; p=0.2429).

Numerically greater mean reductions from baseline in MAGE of CGM glucose were observed for the cotadutide group compared with the placebo group at Day 7 (−25.74 and −13.46 mg/dL, respectively) and Day 14 (−26.59 and 18.05 mg/dL, respectively). On Day 28, a statistically significantly greater reduction from baseline in MAGE of CGM glucose was observed for the cotadutide group compared with the placebo group (LS mean difference: −24.83 mg/dL; p=0.0011).

Changes from baseline in the percentages of 24-hour CGM glucose readings that fell within the euglycemic, hyperglycemic, hypoglycemic, and clinically significant hypoglycemic ranges were assessed at the end of each dosing level.

The euglycemic range was defined as plasma glucose≥70 mg/dL (≥3.9 mmol/L) and ≤180 mg/dL (≤10.0 mmol/L). The mean baseline percentages of CGM glucose readings that fell within the euglycemic range were 82.94% for the cotadutide group and 84.77% for the placebo group. Numerically greater mean increases from baseline in the percentages of CGM glucose readings within the euglycemic range were observed for the cotadutide group compared with the placebo group at Day 7 (7.12% and −5.61%, respectively) and Day 14 (5.31% and −2.79%, respectively). On Day 28, no meaningful difference from baseline in the percentage of 24-hour CGM glucose readings within the euglycemic range was observed for the cotadutide group compared with the placebo group (LS mean difference: 8.78%; p=0.0828).

The hyperglycemic range was defined as plasma glucose>180 mg/dL (>10.0 mmol/L). The mean baseline percentages of CGM glucose readings that fell within the hyperglycemic range were 14.11% for the cotadutide group and 12.24% for the placebo group. Numerically greater mean reductions from baseline in percentages of CGM glucose readings within the hyperglycemic range were observed for the cotadutide group compared with the placebo group at Day 7 (−13.99% and 3.62%, respectively) and Day 14 (−9.81% and 0.36%, respectively). On Day 28, a statistically significantly greater reduction from baseline in the percentage of CGM glucose readings within the hyperglycemic range was observed for the cotadutide group compared with the placebo group (LS mean difference: −12.83%; p=0.0088).

The hypoglycemic range was defined as plasma glucose<70 mg/dL (<3.9 mmol/L). The mean baseline percentages of CGM glucose readings that fell within the hypoglycemic range were 2.61% for the cotadutide group and 2.67% for the placebo group. A numerically greater increase from baseline in the mean percentage of CGM glucose readings within the hypoglycemic range was observed for the cotadutide group compared with the placebo group at Day 7 (6.08% and 1.17%, respectively), and changes from baseline were similar between groups at Day 14 (3.44% and 2.00%, respectively). At Day 28, no meaningful difference from baseline in the percentage of 24 hour CGM glucose readings within the hypoglycemic range was observed for the cotadutide group compared with the placebo group (LS mean difference: 3.35%; p=0.2204).

The clinically significant hypoglycemic range was defined as plasma glucose<54 mg/dL (3.0 mmol/L). The mean baseline percentages of CGM glucose readings that fell within the clinically significant hypoglycemic range were low in both groups (0.58% for the cotadutide group and 0.39% for the placebo group). No meaningful differences from baseline in the percentages of 24 hour CGM glucose readings within the clinically significant hypoglycemic ranges were observed between groups at Day 7, Day 14, or Day 28 (LS mean difference at Day 28: 1.03%; p=0.3692).

In addition, a post-hoc analysis of CGM mean glucose over 7 days was conducted to further evaluate the effect of cotadutide on glucose control at each dose level. Seven-day CGM mean glucose data are summarized below for each 7-day dosing interval (Days 1 to 7 [100 μg dose], Days 8 to 14 [200 μg dose], Days 15 to 21 and Days 22 to 28 [300 μg dose for both]. Statistically significantly greater reductions in CGM mean glucose values over 7 days were observed for the cotadutide group compared with the placebo group at each dose level (Table 6).

TABLE 6
Change in 7-Day CGM Mean Glucose at
Each Dosing Level (ITT Population)

| | | |
|---|---|---|
| | Placebo | Cotadutide |
| Glucose (mg/dL) | N = 24 | N = 25 |

Days 1 to 7 (100 μg dose)

| n | 16 | | 19 | |
|---|---|---|---|---|
| Mean (SD) | 160.08 | (30.39) | 116.82 | (29.51) |
| Median (Min, Max) | 163.26 | (104.5, 216.0) | 111.40 | (72.1, 186.6) |

| | | |
|---|---|---|
| 95% CI | 143.88, 176.27 | 102.59, 131.05 |
| p-value ^{a} | — | 0.0002 |

Days 8 to 14 (200 μg dose)

| n | 16 | | 15 | |
|---|---|---|---|---|
| Mean (SD) | 155.31 | (41.23) | 118.78 | (32.06) |
| Median (Min, Max) | 142.92 | (92.7, 239.3) | 113.48 | (76.1, 174.1) |

| | | |
|---|---|---|
| 95% CI | 133.34, 177.28 | 101.02, 136.53 |
| p-value ^{a} | — | 0.0100 |

Days 15 to 21 (300 μg dose)

| n | 17 | | 18 | |
|---|---|---|---|---|
| Mean (SD) | 151.46 | (32.07) | 120.94 | (34.15) |
| Median (Min, Max) | 150.36 | (96.9, 213.9) | 106.17 | (77.1, 180.5) |

| | | |
|---|---|---|
| 95% CI | (134.96, 167.95) | (103.96, 137.93) |
| p-value ^{a} | | 0.0102 |

Days 22 to 28 (300 μg dose)

| n | 19 | | 22 | |
|---|---|---|---|---|
| Mean (SD) | 155.38 | (39.66) | 115.61 | (34.14) |
| Median (Min, Max) | 145.11 | (93.7, 235.1) | 108.78 | (72.0, 188.7) |

| | | |
|---|---|---|
| 95% CI | 136.27, 174.50 | 100.47, 130.75 |
| p-value ^{a} | — | 0.0016 |

CGM = continuous glucose monitoring;
CI = confidence interval;
ITT = intent-to-treat;
Max = maximum;
Min = minimum;
SD = standard deviation
Note:
Subjects were on metformin and dapagliflozin background dual therapy during the double-blind treatment period.
^{a} P-value from 2-sample t-test.

### Change in FPG

The change in fasting plasma glucose (FPG) from baseline to Day 28 was assessed. A statistically significantly greater reduction from baseline to Day 28 in FPG was observed for the cotadutide group compared with the placebo group (LS mean difference: −37.73 mg/dL; p<0.0001) (Table 7).

TABLE 7
Change from Baseline to Day 28 in Fasting
Plasma Glucose (ITT Population)

| | | |
|---|---|---|
| Fasting Plasma | Placebo | Cotadutide |
| Glucose (mg/dL) | N = 24 | N = 25 |

Baseline

| n | 24 | | 25 | |
|---|---|---|---|---|
| Mean (SD) | 148.82 | (26.28) | 149.57 | (26.58) |
| Median (Min, | 145.06 | (108.1, 219.8) | 140.56 | (120.7, 239.7) |
| Max) | | | | |

Day 28 (LOCF)

| n | 24 | | 25 | |
|---|---|---|---|---|
| Mean (SD) | 152.49 | (36.06) | 115.33 | (30.78) |
| Median (Min, | 152.27 | (84.7, 205.4) | 106.32 | (81.1, 200.0) |
| Max) | | | | |

Change from baseline (LOCF)

| n | 24 | | 25 | |
|---|---|---|---|---|
| Mean (SD) | 3.68 | (26.83) | −34.24 | (28.67) |
| Median (Min, | 4.51 | (−45.1, 75.7) | −43.25 | (−82.9, 61.3) |
| Max) | | | | |

| | | |
|---|---|---|
| LS Mean ^{a} | 3.58 | −34.15 |
| 95% CI of LS | −7.62, 14.79 | (−45.13, −23.17) |
| Mean ^{a} | | |
| LS mean | — | −37.73 |
| difference ^{a} | | |
| 95% CI ^{a} | — | −53.42, −22.04 |
| p-value ^{a} | — | <0.0001 |

ANCOVA = analysis of covariance;
CI = confidence interval;
ITT = intent-to-treat;
LOCF = last observation carried forward;
LS = least squares;
Max = maximum;
Min = minimum;
SD = standard deviation
Note:
Subjects were on metformin and dapagliflozin background dual therapy during the double-blind treatment period.
^{a} LS Mean, its associated 95% CI, and p-value from ANCOVA with treatment effect and baseline value as covariate.

### Change in HbA1c

The change in hemoglobin A1c (glycated hemoglobin; HbA1c) from baseline to Day 28 was assessed. A statistically significantly greater reduction from baseline to Day 28 in HbA1c was observed for the cotadutide group compared with the placebo group (LS mean difference: −0.58%; <0.0001) (Table 8).

TABLE 8
Change from Baseline to Day 28 in
Hemoglobin A1c (ITT Population)

| | | |
|---|---|---|
| | Placebo | Cotadutide |
| Hemoglobin A1c | N = 24 | N = 25 |

Baseline (%)

| | | |
|---|---|---|
| n | 24 | 25 |

| | | | | |
|---|---|---|---|---|
| Mean (SD) | 7.60 | (0.80) | 7.78 | (0.83) |
| Median (Min, Max) | 7.45 | (6.5, 9.9) | 7.60 | (6.5, 10.1) |

Day 28 (%)

| | | |
|---|---|---|
| n | 23 | 24 |

| | | | | |
|---|---|---|---|---|
| Mean (SD) | 7.21 | (0.74) | 6.75 | (0.71) |
| Median (Min, Max) | 7.10 | (6.2, 9.0) | 6.65 | (5.6, 8.7) |

Change from baseline

| | | |
|---|---|---|
| n | 23 | 24 |

| | | | | |
|---|---|---|---|---|
| Mean (SD) | −0.43 | (0.28) | −1.04 | (0.42) |
| Median (Min, Max) | −0.40 | (−0.9, 0.1) | −1.10 | (−1.9, 0.0) |

| | | |
|---|---|---|
| LS Mean ^{a} | −0.45 | −1.03 |
| 95% CI of LS Mean ^{a} | −0.58, −0.31 | −1.16, −0.90 |
| LS mean difference ^{a} | — | −0.58 |
| 95% CI ^{a} | — | −0.77, −0.39 |
| p-value ^{a} | — | <0.0001 |

ANCOVA = analysis of covariance;
CI = confidence interval;
ITT = intent-to-treat;
LS = least squares;
Max = maximum;
Min = minimum;
SD = standard deviation
Note:
Subjects were on metformin and dapagliflozin background dual therapy during the double-blind treatment period.
^{a} LS Mean, its associated 95% CI, and p-value from ANCOVA with treatment effect and baseline value as covariate.

### Change in Body Weight

The change and percentage change in body weight from baseline to Day 29 were assessed. Statistically significantly greater reductions from baseline to Day 29 were observed for the cotadutide group compared with the placebo group in body weight (LS mean difference: −2.13 kg; p=0.0002) and percentage body weight (LS mean difference: −2.26%; p=0.0002) (Table 9).

TABLE 9
Change and Percent Change in Body Weight
from Baseline to Day 29 (ITT Population)

| | Placebo | | Cotadutide | |
|---|---|---|---|---|
| Weight (kg) | N = 24 | | N = 25 | |
| Baseline, n | 24 | | 25 | |
| Mean (SD) | 99.63 | (16.87) | 92.24 | (14.19) |
| Median (Min, Max) | 95.00 | (74.3, 134.9) | 89.60 | (63.0, 127.6) |
| Day 29 change from | 24 | | 24 | |
| baseline, n | | | | |
| Mean (SD) | −1.14 | (1.49) | −3.06 | (2.12) |
| Median (Min, Max) | −1.05 | (−4.4, 2.1) | −2.85 | (−8.9, 1.3) |
| LS mean ^{a} | −1.03 | | −3.16 | |
| 95% CI of LS mean ^{a} | −1.78, −0.28 | | −3.91, −2.42 | |
| LS mean difference ^{a} | — | | −2.13 | |
| 95% CI ^{a} | — | | −3.20, −1.06 | |
| p-value ^{a} | — | | 0.0002 | |
| Day 29 percent change | 24 | | 24 | |
| from baseline (%), n | | | | |
| Mean (SD) | −1.10 | (1.50) | −3.30 | (2.19) |
| Median (Min, Max) | −1.11 | (−3.9, 2.5) | −3.14 | (−9.9, 1.5) |

| | | |
|---|---|---|
| LS mean ^{a} | −1.07 | −3.33 |
| 95% CI of LS mean ^{a} | −1.86, −0.29 | −4.12, −2.54 |
| LS mean difference ^{a} | — | −2.26 |
| 95% CI ^{a} | — | −3.39, −1.13 |
| p-value ^{a} | — | 0.0002 |

ANCOVA = analysis of covariance;
CI = confidence interval;
ITT = intent-to-treat;
LOCF = last observation carried forward;
LS = least squares
Note:
Subjects were on metformin and dapagliflozin background dual therapy during the double-blind treatment period.
^{a} LS Mean, its associated 95% CI, and p-value from ANCOVA with treatment effect and baseline value as covariate.

The proportion of subjects achieving>5% body weight loss from baseline to Day 29 was also assessed. No subjects in the placebo group and 3 subjects (12.5%) in the cotadutide groups achieved>5% body weight loss from baseline to Day 29.

### Pancreatic and Incretin Hormone Profiles During the MMTT

The changes in insulin and C-peptide AUC₀₀₄ₕ from baseline to Day 28 were assessed. At baseline, the mean insulin AUC₀₋₄ h was higher in the cotadutide group compared with the placebo group (91.768 vs 78.472 hr·mU/L, respectively). There was no meaningful difference from baseline in LS mean insulin AUC₀₋₄ h at Day 28 between the cotadutide and placebo groups (−0.08 and 2.78 hr·mU/L, respectively; LS mean difference: −2.86 hr·mU/L; p=0.7760). At baseline, the mean C-peptide AUC₀₋₄ h was similar in the cotadutide and placebo groups (11.278 vs 10.605 hr. μg/L, respectively). There was no meaningful difference from baseline in LS mean C-peptide AUC₀₋₄ h at Day 28 between the cotadutide and placebo groups (0.32 and −0.29 hr. μg/L, respectively (LS mean difference: 0.61 hr·mU/L; p=0.4345).

Changes in glucagon AUC₀₋₄ h from baseline to Day 28 were assessed, and there was no meaningful difference from baseline in LS mean glucagon AUC₀₋₄ h at Day 28 between the cotadutide and placebo groups (−12.52 and −23.84 hr.ng/L, respectively; LS mean difference: 11.32 hr.pg/L; p=0.7687).

Changes in GLP-1 (active form) AUC₀₋₄ h from baseline to Day 28 were assessed, and there was no meaningful difference from baseline in LS mean GLP-1 AUC₀₋₄ h at Day 28 between the cotadutide and placebo groups (−131.67 and −130.93 hr.ng/L, respectively; LS mean difference: −0.73 hr.ng/L; p=0.9946).

Ketone Bodies and FFA levels

The changes in fasting free fatty acid (FFA) from baseline to the end of each dose level were assessed. Fasting FFA changes from baseline to Day 7 (100 μg), Day 14 (200 μg), or Day 28 (300 μg) were similar between treatment groups (LS mean difference at Day 28: −0.04 mEq/L; p=0.5322).

### Glucose Urine Excretion

Changes in 24-hour glucose urine excretion from baseline to Day 28 were assessed. At baseline, subjects in the cotadutide group had less urine glucose excretion compared with subjects in the placebo group (4552.85 vs 9107.06 mg/24 hr, respectively). There was a smaller reduction from baseline in LS mean glucose urine excretion at Day 28 in the cotadutide group compared with the placebo group (−643.46 vs −1564.72 mg/24h, respectively); however, the difference between groups was not statistically significant (LS mean difference: 921.26 mg/24h; p=0.4128).

### Pharmacokinetic Parameters

The endpoints of Cₘₐₓ, tₘₐₓ, t_{1/2}, Cl/F, AUC_{0-inf}, AUC₀₋ₗₐₛₜ, and AUC_{τ} were assessed to evaluate the PK profile of cotadutide and dapagliflozin (in subjects treated with dapagliflozin, metformin, and cotadutide) and dapagliflozin (in subjects treated with dapagliflozin, metformin, and placebo).

The Cₘₐₓ and the corresponding tₘₐₓ were identified from the observed data. Log-linear regression of the terminal phase yielded the terminal rate constant (λ_{z}). The area under the plasma concentration time-curve during the dosing interval (AUC_{τ}) was calculated by the log-linear trapezoidal rule. The area under the curve to infinity (AUC_{0-inf}) was calculated as the sum of AUC(ₒ₋ₜ) and Ct/z, where Ct is the observed plasma concentration obtained from the log-linear regression analysis of the last quantifiable time point and z is the terminal phase rate constant. Clearance (CL/F) was determined by dose/AUC_{0-inf}, and the terminal half-life (t_{1/2}) was calculated as 0.693/λ_{z}.

Mean PK parameters of dapagliflozin alone were similar to those reported in literature (Dapagliflozin New Drug Application submission 202293. FDA. 11Jul. 2013): Cₘₐₓ of 120 ng/mL, tₘₐₓ of 1 hour, AUCₘf of 516 to 506 ng.hr/mL, half-life of 15 hours, and apparent clearance of approximately 20 L/hr for doses of 10 mg/day per os (orally; PO). A slightly higher apparent clearance was derived for dapagliflozin in this study (in the range of 25 L/hr) in both treatment groups.

Maximal plasma concentration of dapagliflozin was impacted in the presence of cotadutide: a mean Cₘₐₓ of 116.7 ng/mL was observed on Day −1, decreasing to 84.4 ng/mL and 61.1 ng/mL on Days 7 and 14, respectively, then increasing again to 94.2 ng/mL by Day 28. A similar effect was observed in the group receiving dapagliflozin and placebo, where the mean Cₘₐₓ observed was 110.1 ng/mL on Day −1, decreasing to 92.0 ng/mL and 95.6 ng/mL on Days 7 and 14 respectively, then increased again to 112.2 ng/mL by Day 28. Similar variability also was observed across different occasions between Day −1 and Day 28 on Cₘₐₓ (CV % on Cₘₐₓ in the range of 35 to 55% for both treatments, with and without cotadutide). This impact could result from the effect of cotadutide on gastric emptying.

A slightly higher overall dapagliflozin exposure was seen across all occasions, in terms of AUC_{inf}, in the treatment group where dapagliflozin was co-administered with cotadutide, although this was observed as early as Day −1 (when cotadutide was not yet co-administered). The analysis of dapagliflozin C_{trough}h plasma concentrations also confirmed a minimal increase in the pre-dose concentrations of dapagliflozin in co-administration with cotadutide; nevertheless, similar accumulation ratios for dapagliflozin between the 2 treatment groups were calculated, suggesting no major changes in the overall daily exposure driven by the presence of cotadutide.

A slightly higher apparent clearance (approximately 25 L/hr) compared with literature data (20 L/hr) and consequently lower t_{1/2} (approximately 8 hours compared with 15 hours) was derived in both treatment groups consistently across all 4 occasions, which therefore could not be associated to the co-administration of dapagliflozin with cotadutide.

Cotadutide PK in the presence of dapagliflozin was in line with historical data for this compound: a linear Cₘₐₓ, was observed in the dose range tested (mean Cₘₐₓ of 5.2 ng/mL, 10.1 ng/mL and 17.2 ng/mL at the doses of 100, 200 and 300 μg, respectively) as well as AUC_{inf} (mean AUC_{inf} of 106.4 ng.hr/mL, 196.7 ng.hr/mL, and 314.6 ng.hr/mL at the doses of 100, 200 and 300 μg, respectively). Apparent clearance and half-life were also consistent across doses (CL/F of 1.1 to 1.3 L/hr and t_{1/2} of 8.8 to 9.1 hours) and were in line with historical data.

Plasma levels of β-hydroxybutyrate were similar across both treatments in all occasions. Average maximal concentrations (Cₘₐₓ) of ketones observed in subjects treated with dapagliflozin alone ranged from 0.29 to 0.35 mmol/L compared to 0.31 to 0.39 mmol/L observed in subjects treated with dapagliflozin in combination with cotadutide between Days−1 and 28. Similarly, average daily exposure as measured by AUC_{τ} was in the range of 4.58 to 5.32 mmol·hr/L in subjects treated with dapagliflozin alone compared to 4.95 to 5.44 mmol·hr/L observed in subjects treated with dapagliflozin in combination with cotadutide between Days −1 and 28.

Analysis of cotadutide predose plasma concentrations (C_{trough}) showed increasing mean concentrations of 1.9, 3.5, and 5.2 ng/mL on Days 7, 14, and 28 respectively, in agreement with the dose titration of 100, 200, and 300 μg dose and overall confirming PK linearity of the peptide as from historical data.

Overall, PK results of dapagliflozin 10 mg/day PO alone were in line with literature results, and PK results of cotadutide 100 to 300 μg/day were in line with historical data. cotadutide clinical PK was not affected by the presence of dapagliflozin, and a marginal impact on dapagliflozin oral absorption was observed, likely related to the delay in gastric emptying driven by GLP-1 pharmacology.

### Immunogenicity

Development of ADA and titres (in any subjects testing positive) were assessed to evaluate the immunogenicity profile of cotadutide titrated up to a dose of 300 μg. No subjects in the placebo group and 3 subjects (12.0%) in the cotadutide group tested positive for ADA at baseline. None of these subjects who tested positive at baseline were ADA positive post-baseline. One subject (4.2%) in the placebo group and 3 subjects (12.5%) in the cotadutide group were ADA positive post-baseline. No subjects had a treatment boosted ADA, defined as baseline ADA titre that was boosted to a 4-fold or higher level during drug administration.

By visit, no subjects in the placebo group and 1 subject (4.2%) in the cotadutide group were ADA positive at Day 29. One subject (4.2%) in the placebo group and 2 subjects (8.3%) in the cotadutide group were ADA positive at the End of Study.

### Safety

Overall, the incidence of treatment-emergent adverse events (TEAEs) was similar in the cotadutide and placebo groups (52.0% and 58.3%, respectively). Two subjects (both in the cotadutide group) reported a TEAE that led to discontinuation of investigational product (due to events of abdominal pain and vomiting). All TEAEs were mild (Grade 1) or moderate (Grade 2) in severity.

Investigational product-related TEAEs (as judged by the investigator) were more frequent in the cotadutide group when compared with the placebo group (40.0% and 16.7%, respectively). The majority of TEAEs related to cotadutide treatment were in the SOC of Gastrointestinal disorders.

The incidence of dapagliflozin-related TEAEs (as judged by the investigator) was low and not notably different in the cotadutide and placebo groups (4.0% and 8.3%, respectively).

There were no clinically meaningful trends in hematology, serum chemistry, or urinalysis laboratory values following administration of cotadutide. No meaningful changes in blood pressure (BP) were observed as a result of cotadutide administration; however, increased heart rate was observed in the cotadutide group compared with the placebo group. With the exception of heart rate, no clinically meaningful trends in quantitative or qualitative electrocardiogram (ECG) parameters were observed.

### CONCLUSIONS

Statistically significantly greater reductions in change and percentage change from baseline to Day 28 in MMTT plasma glucose AUC₀₋₄ h were observed in the cotadutide group compared with the placebo group.

Reductions in CGM glucose AUC₀₋₂₄ h and mean glucose over 24 hours were observed in the cotadutide group titrated weekly up to a dose level of 300 μg compared with the placebo group beginning at Day 7 and continuing through Day 28. Improvements in glycemic variability, as assessed by SD and MAGE of CGM glucose values over 24 hours, were observed at all dose levels; no meaningful differences in CV of CGM glucose were observed between groups.

The percentage of CGM glucose readings within the euglycemic range was high at baseline in both treatment groups, as was expected for subjects on dual background treatment with dapagliflozin and metformin. Numerically greater increases in the percentage of CGM glucose readings within the euglycemic range from baseline were observed in the cotadutide group compared with the placebo group at the end of each dosing level. A statistically significantly greater reduction in the percentage of CGM glucose readings within the hyperglycemic range was observed in the cotadutide group compared with the placebo group at the end of 28 days of dosing. No meaningful differences in the percentages of CGM glucose readings within the hypoglycemic or clinically significant hypoglycemic ranges were observed at any dose level.

Statistically significantly greater reductions in CGM mean glucose values over 7 days were observed for the cotadutide group compared with the placebo group at each dose level.

Statistically significantly greater reductions from baseline to the end of 28 days of treatment were observed in the cotadutide group compared with the placebo group in FPG and HbA1c.

A statistically significantly greater reduction from baseline to the end of 29 days of treatment was observed in the cotadutide group compared with the placebo group in body weight. No subjects in the placebo group and 3 subjects (12.5%) in the cotadutide groups achieved>5% body weight loss from baseline to Day 29.

There were no meaningful differences between the cotadutide and placebo groups in pancreatic and incretin hormone profiles including insulin, C-peptide, glucagon, and GLP-1 (active form) during MMTT at the end of 28 days of treatment.

Similar fasting FFA changes from baseline to the end of each dosing level were observed in the cotadutide and placebo groups. There were no meaningful differences between the cotadutide and placebo groups in β-hydroxybutyrate levels (ketone bodies).

There was not a statistically significant difference between groups in 24-hour urine glucose excretion after 28 days of treatment.

***

The disclosure is not to be limited in scope by the specific embodiments described which are intended as single illustrations of individual aspects of the disclosure, and any compositions or methods which are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

What is claimed is:

1. A method of improving glycemic control in a human patient in need thereof, the method comprising administering to the patient a sufficient amount of:
(i) cotadutide (SEQ ID NO:4);
(ii) dapagliflozin; and
(iii) metformin to improve glycemic control.

2. A method of reducing body weight in a human patient in need thereof, the method comprising administering to the patient a sufficient amount of:
(i) cotadutide (SEQ ID NO:4);
(ii) dapagliflozin; and
(iii) metformin to reduce body weight.

3. A method of treating type 2 diabetes mellitus (T2DM) in a human patient in need thereof, the method comprising administering to the patient a sufficient amount of:
(i) cotadutide (SEQ ID NO:4);
(ii) dapagliflozin; and
(iii) metformin to treat T2DM.

4. The method of any one of claims 1-3, wherein the cotadutide is administered at an initial dose of at least 20 μg daily and administered at a second higher dose thereafter.

5. The method of claim 4, wherein the cotadutide is administered at a third dose after the administration of the second dose, wherein the third dose is higher than the second dose, optionally wherein the third dose does not exceed 600 μg daily or wherein the third dose does not exceed 300 μg daily.

6. The method of claim 4 or 5, wherein the initial dose is administered for about 7 days to about 14 days.

7. The method of any one of claims 1-6, wherein the cotadutide is administered at an initial dose of 100 μg daily for 7 days, at second dose of 200 μg daily for the next 7 days, and subsequently at a dose of 300 μg daily.

8. The method of any one of claims 1-7, wherein the cotadutide is administered by injection, optionally wherein the administration is subcutaneous.

9. The method of any one of claims 1-8, wherein the dapagliflozin is administered at a dose of 5 mg or 10 mg daily, optionally at a dose of 10 mg daily.

10. The method of any one of claims 1-9, wherein the dapagliflozin is administered orally.

11. The method of any one of claims 1-10, wherein the metformin is administered at a dose of 500 mg to 2550 mg daily, 500 mg to 2000 mg daily, 500 mg to 1000 mg daily, or 500 mg to 850 mg daily.

12. The method of any one of claims 1-11, wherein the metformin is administered orally.

13. The method of any one of claims 1-12, wherein the administration reduces the mixed-meal tolerance test (MMTT) plasma glucose area under the curve (AUC)₀₋₄ hours in the patient.

14. The method of claim 13, wherein the administration reduces the MMTT plasma glucose AUC₀₋₂₄ hours in the patient by at least 25 mg-hr/dL, at least 50 mg-hr/dL, at least 75 mg-hr/dL, at least 100 mg-hr/dL, or at least 150 mg-hr/dL.

15. The method of claim 13 or 14, wherein the administration reduces the percent MMTT plasma glucose AUC₀₋₂₄ hours in the patient by at least 5%, at least 10%, at least 15%, or at least 20%.

16. The method of any one of claims 1-15, wherein the administration reduces continuous glucose monitoring (CGM) glucose AUC₀₋₂₄ in the patient.

17. The method of claim 16, wherein the administration reduces CGM glucose AUC₀₋₂₄ in the patient by at least 200 mg-hr/dL, at least 250 mg-hr/dL, at least 300 mg-hr/dL, at least 350 mg-hr/dL, at least 400 mg-hr/dL, at least 450 mg-hr/dL, at least 500 mg-hr/dL, at least 550 mg-hr/dL, at least 600 mg-hr/dL, or at least 650 mg-hr/dL

18. The method of any one of claims 1-17, wherein the administration reduces 24-hour CGM mean glucose in the patient.

19. The method of claim 18, wherein the administration reduces 24-hour CGM mean glucose in the patient by at least 10 mg/dL, at least 15 mg/dL, at least 20 mg/dL, or at least 25 mg/dL.

20. The method of any one of claims 1-19, wherein the administration reduces the standard deviation (SD) in CGM glucose in the patient by at least 5 mg/dL.

21. The method of any one of claims 1-20, wherein the administration reduces the CGM mean amplitude of glucose excursion (MAGE) in the patient by at least 10 mg/dL, at least 15 mg/dL, at least 20 mg/dL, or at least 25 mg/dL.

22. The method of any one of claims 1-21, wherein the administration reduces fasting plasma glucose (FPG) in the patient.

23. The method of claim 22, wherein the administration reduces FPG in the patient by at least 5 mg/dL, at least 10 mg/dL, at least 15 mg/dL, at least 20 mg/dL, at least 25 mg/dL, or at least 30 mg/dL.

24. The method of any one of claims 1-23, wherein the administration reduces Hemoglobin A1c (HbA1c) in the patient by at least 0.5% or by at least 1%.

25. The method of any one of claims 1-24, wherein the administration reduces body weight of the patient by at least 2 kg or by at least 3 kg.

26. The method of any one of claims 13-25, wherein the reduction occurs with 28 days from the initial administration of the cotadutide.

27. The method of any one of claims 1-26, wherein the administration produces euglycemic glucose levels in the patient.

28. The method of any one of claims 1-27, wherein the administration prevents hyperglycemic glucose levels in the patient.

29. The method of any one of claims 2-28, wherein the administration improves glycemic control in the patient.

30. The method of any one of claims 1 and 3-28, wherein the administration reduces body weight in the patient.

31. The method of any one of claims 1, 2, and 4-28, wherein the administration treats T2DM in the patient.

32. The method of any one of claims 1-31, wherein the administration is for at least four weeks.

33. The method of any one of claims 1-32, wherein the administration is an adjunct to diet and exercise.

34. The method of any one of claims 1-33, wherein the patient has a body mass index (BMI) of ≥25 kg/m² to ≤40 kg/m².

35. The method of any one of claims 1-34, wherein the patient has a hemoglobin A1c (HbA1c) of ≥7.0% to ≤10.0%.

36. The method of any one of claims 1, 2, 4-30, and 32-35, wherein the patient has T2DM.


SEQUENCE LISTING (4 sequences)

SEQ ID NO: 1 - protein, 29 aa, Homo sapiens
HSQGTFTSDYSKYLDSRRAQDFVQWLMNT

SEQ ID NO: 2 - protein, 30 aa, Homo sapiens
HAEGTFTSDVSSYLEGQAAKEFIAWLVKGR

SEQ ID NO: 3 - protein, 31 aa, Homo sapiens
HAEGTFTSDVSSYLEGQAAKEFIAWLVKGRG

SEQ ID NO: 4 - protein, 30 aa, Artificial Sequence
  chimeric GLP-1/glucagon peptide
  MISC_FEATURE (10)..(10): Lysine with a palmitoyl group conjugated to the epsilon nitrogen,through a gamma glutamic acid linker
HSQGTFTSDKSEYLDSERARDFVAWLEAGG